(19) United States
(12) Patent Application Publication
(10) Pub. No.: US 2025/0127583 A1
(43) Pub. Date: Apr. 24, 2025

(54) A MICROMANIPULATOR AND SYSTEM

(71) Applicant: NANYANG TECHNOLOGICAL UNIVERSITY, Singapore (SG)

(72) Inventors: Wei Tech ANG, Singapore (SG); Chao ZHOU, Singapore (SG); Chen FENG, Singapore (SG); Kwang Yong LIM, Singapore (SG); Zuping JIANG, Singapore (SG); Yuan YUAN, Singapore (SG)

(21) Appl. No.: 18/691,956

(22) PCT Filed: Oct. 21, 2022
(86) PCT No.: PCT/SG2022/050753

§ 371 Date: Mar. 14, 2024

(30) Foreign Application Priority Data
Oct. 21, 2021 (SG) 10202111705R
Oct. 21, 2021 (SG) 10202111710W

(51) Int. Cl. A61B 34/00; A61B 34/20; A61B 90/00; A61B 90/20

(52) U.S. Cl. CPC A61B 34/72; A61B 34/75; A61B 90/20; A61B 2034/2057; A61B 2090/3945

(57) ABSTRACT

A micromanipulator comprising: a first frame portion defining an axial direction extending from a first proximal end to a first distal end; a second frame portion; a tool holder coupled to the second frame portion; a first connecting portion coupling the second frame portion and the first frame portion at the first distal end in resilient pivotable coupling; and a second connecting portion coupling the second frame portion and the first frame portion at the first proximal end in resilient pivotable coupling. The first frame portion, the second frame portion, the first connecting portion, and a second connecting portion form a flexural frame. The flexural frame is resiliently biased to a first frame state in which the tool holder is in a retracted position.

The present application claims priority to the Singapore patent applications no. 10202111705R and 10202111710W which are incorporated in their entirety by reference.

### TECHNICAL FIELD

The present disclosure relates to the field of medical devices, and more particularly to tremor-mitigating handheld instruments.

### BACKGROUND

The precision of a handheld instrument in various applications such as, but not limited to, supermicrosurgical procedures, is dependent on the user's control over the user's own movement and/or posture, etc. However, the human hand, even of a healthy and trained user, may exhibit tiny involuntary movements. These involuntary movements may be the result of physiological tremor, myoclonia, low-frequency drifts, etc.

### SUMMARY

In one aspect, the present application discloses a micromanipulator. The micromanipulator comprises: a first frame portion, the first frame portion defining an axial direction extending from a first proximal end to a first distal end; a second frame portion; a tool holder, the tool holder being coupled to the second frame portion; a first connecting portion, the first connecting portion coupling the second frame portion and the first frame portion at the first distal end in resilient pivotable coupling; and a second connecting portion, the second connecting portion coupling the second frame portion and the first frame portion at the first proximal end in resilient pivotable coupling, wherein the first frame portion, the second frame portion, the first connecting portion, and a second connecting portion form a flexural frame, the flexural frame being resiliently biased to a first frame state in which the tool holder is in a retracted position. The retracted position may be relative to the first distal end.

The micromanipulator, further comprising a primary actuator coupled to the first frame portion, the primary actuator being configured to provide a push force in the axial direction on the first connecting portion to angularly displace the first connecting portion about a first axis relative to the first frame portion, the first axis being orthogonal to the axial direction.

The micromanipulator in which a displacement of the second frame portion responsive to an actuation of the primary actuator results in a reduced footprint of the flexural frame.

The micromanipulator further comprising: a first secondary actuator coupled to the second frame portion, the first secondary actuator being a linear actuator having a first secondary actuator axis; and a second secondary actuator coupled to the second frame portion, the second secondary actuator being a linear actuator having a second secondary actuator axis, wherein the primary actuator is a linear actuator having a primary actuator axis, and wherein the primary actuator axis, the first secondary actuator axis, and the second secondary axis are non-coincident with one another.

The micromanipulator in which the primary actuator axis, the first secondary actuator axis, and the second secondary axis may be parallel to the axial direction at least when the flexural frame is in the first frame state. The micromanipulator in which each of the first secondary actuator and the second secondary actuator may be configured to provide a push force on the tool holder. The tool holder may be angularly displaced relative to the second frame portion in response to actuation of at least one of the first secondary actuator and the second secondary actuator.

The micromanipulator further comprising a controller, the controller being configured to be in signal communication with the primary actuator, the first secondary actuator, and the second secondary actuator, wherein the controller may be configured to controllably actuate each of the primary actuator, the first secondary actuator, and the second secondary actuator.

The micromanipulator, wherein responsive to the first connecting portion pivoting relative to the first frame portion about a first axis, the second frame portion may be displaced in the axial direction, wherein the first axis is orthogonal to the axial direction. The micromanipulator, wherein responsive to an angular displacement of the first connecting portion relative to the first frame portion about the first axis, the second frame portion may be displaced along a lateral direction towards the first frame portion, wherein the lateral direction is orthogonal to both the first axis and the axial direction.

The micromanipulator in which the flexural frame may be integrally formed. The micromanipulator may further comprise a housing, the housing being coupled to the first frame portion, wherein the micromanipulator is a handheld instrument. The micromanipulator may further comprise at least one marker coupled to an exterior of the housing. The micromanipulator may further comprise a plurality of markers coupled to an exterior of the housing, wherein each of the at least three markers are configured to emit light.

In another aspect, the present application discloses a system, comprising: a tool; a micromanipulator, the micromanipulator including: a first frame portion, the first frame portion defining an axial direction extending from a first proximal end to a first distal end; a second frame portion; a tool holder, the tool holder being coupled to the second frame portion, the tool being releasably attached to the tool holder; a first connecting portion, the first connecting portion coupling the second frame portion and the first frame portion at the first distal end in resilient pivotable coupling; a second connecting portion, the second connecting portion coupling the second frame portion and the first frame portion at the first proximal end in resilient pivotable coupling, wherein the first frame portion, the second frame portion, the first connecting portion, and a second connecting portion form a flexural frame, the flexural frame being resiliently biased to a first frame state in which the tool holder is in a retracted position relative to the first distal end; a housing coupled to the first frame portion; and at least one marker disposed on the housing; and a controller, the controller being in signal communication with the micromanipulator, the controller being configured to determine a tremor-mitigating displacement based on images captured of the at least one marker and to control.

The micromanipulator may further comprise a primary actuator coupled to the first frame portion, the primary actuator being configured to provide a push force in the axial direction on the first connecting portion to angularly displace the first connecting portion about a first axis relative to the first frame portion, the first axis being orthogonal to the axial direction; and two secondary actuators coupled to the second frame portion, each of the two secondary actuators being a linear actuator with a respective secondary actuator axis, wherein the primary actuator axis and the respective second actuator axis are non-coincident with one another, wherein the controller is configured to control the primary actuator and the two secondary actuators to provide a tremor-mitigating displacement of the tool.

The system may comprise a camera configured to capture the images, wherein the camera is a motion capture camera. The at least one marker may be a light emitting diode, in which the camera and the at least one marker are configured to match in frequency. The system may comprise two cameras having a combined camera field of view, wherein the two cameras are mounted to a support arm above a workspace, and support arm being coupled with a microscope having a microscope field of view, and wherein the two cameras are oriented such that the combined camera field of view at least overlaps the microscope field of view.

A system comprising: a tool; a micromanipulator, the micromanipulator including: a tool holder, the tool being releasably attached to the tool holder; a housing; and at least one marker disposed on the housing; and at least one camera, the at least one camera being configured to capture images of the at least one marker; and a controller, the controller being configured to be in signal communication with the micromanipulator, the controller being configured to determine a tremor-mitigating displacement for the tool based on the images of the at least one marker, wherein each one of the at least one camera is a motion capture optical camera having an operational accuracy of at least 400 microns, and wherein the tremor-mitigating displacement is determined to an accuracy of at least 10 microns.

The controller may be configured to determine a position of the tool holder in a workspace, in which the workspace is a three-dimensional space that is at least one order of magnitude smaller than a camera field of view of the at least one camera. The system may further comprise a microscope having a microscope field of view, wherein the at least one camera has a combined camera field of view configured to at least overlap the microscope field of view. The at least one marker may be a light emitting diode, and the at least one camera and the at least one marker may be configured to match in frequency. Each one of the at least one camera is positioned further away from the workspace than an objective lens of the microscope.

### BRIEF DESCRIPTION OF DRAWINGS

FIG. 1A is a schematic perspective view of a robot-assisted surgical (RAS) system according to an embodiment of the present disclosure;

FIG. 1B is a schematic perspective view showing a preferred system according to another embodiment of the present disclosure;

FIG. 2 is a schematic flow chart showing a method of mitigating tremor for a handheld instrument;

FIG. 3 is a perspective view of a micromanipulator coupled with an interchangeable tool;

FIG. 4 is an exploded perspective view of one embodiment of the micromanipulator of the present disclosure;

FIG. 5A is a side view of a flexural frame in a first frame state;

FIG. 5B is a side view of the flexural frame in a second frame state;

FIG. 6 is a front view of FIG. 5A;

FIG. 7 is a front view of FIG. 5B

FIG. 8 is a side view of a flexural frame according to another embodiment in a first frame state;

FIG. 9 is a side view of the flexural frame according to FIG. 8 in a second frame state;

FIG. 10 is a sectional view A-A of a flexural frame according to an embodiment;

FIG. 11 is a partial detailed view of the flexural frame of FIG. 10;

FIG. 12 is a partial detailed sectional view A-A of the flexural frame of FIG. 10 in a first frame state;

FIG. 13 is a partial detailed sectional view A-A of the flexural frame of FIG. 10 in a second frame state;

FIG. 14 is a front view B1-B1 of FIG. 12;

FIG. 15 is a front view B2-B2 of FIG. 13;

FIG. 16 is a partial perspective view of the flexural frame;

FIG. 17 is a sectional view C-C of FIG. 16;

FIG. 18 is a detailed sectional of the flexural frame of FIG. 16 in a neutral position;

FIG. 19 is a detailed sectional of the flexural frame of FIG. 16 in an actuated position.

### DETAILED DESCRIPTION

Reference throughout this specification to “one embodiment”, “another embodiment” or “an embodiment” (or the like) means that a particular feature, structure, or characteristic described in connection with the embodiment is included in at least one embodiment. Thus, the appearance of the phrases “in one embodiment” or “in an embodiment” or the like in various places throughout this specification are not necessarily all referring to the same embodiment. Furthermore, the described features, structures, or characteristics may be combined in any suitable manner in one or more embodiments. In the following description, numerous specific details are provided to give a thorough understanding of embodiments. One skilled in the relevant art will recognize that the various embodiments may be practiced without one or more of the specific details, or with other methods, components, materials, etc. In other instances, some or all known structures, materials, or operations may not be shown or described in detail to avoid obfuscation.

The word “exemplary” is used herein to mean “serving as an example, instance, or illustration.” Any embodiment described herein as “exemplary” is not necessarily to be construed as preferred or advantageous over other embodiments. As used herein, the singular ‘a’ and ‘an’ may be construed as including the plural “one or more” unless apparent from the context to be otherwise.

Terms such as “first” and “second” are used in the description and claims only for the sake of brevity and clarity, and do not necessarily imply a priority or order, unless required by the context. The terms “about” and “approximately” as applied to a stated numeric value encompasses the exact value and a reasonable variance as will be understood by one of ordinary skill in the art, and the terms “generally” and “substantially” are to be understood in a similar manner, unless otherwise specified.

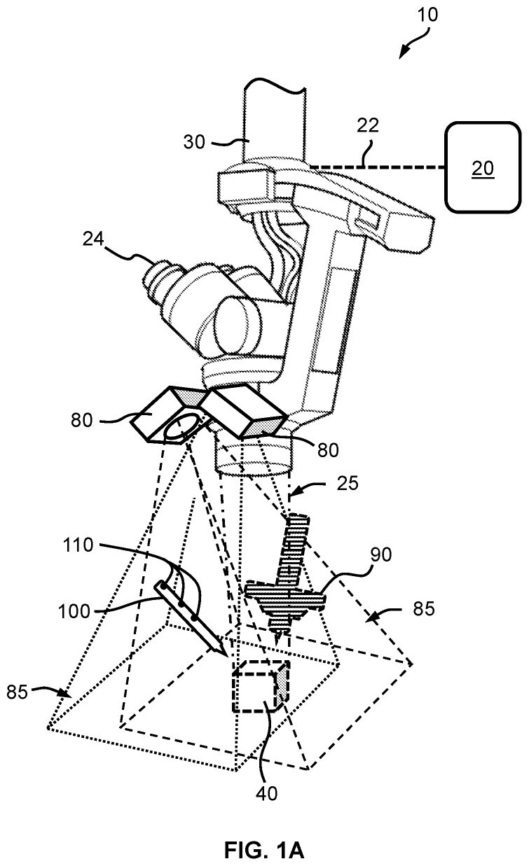

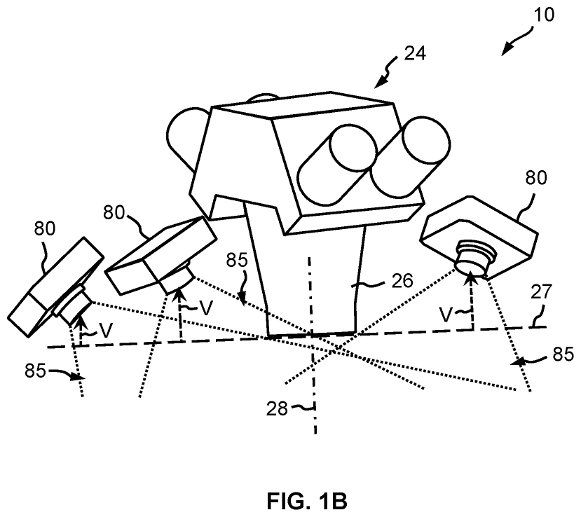

FIG. 1A is a schematic perspective view illustrating one non-limiting example of a system 10. The system 10 may include one or more microscopes 24 for a user to view microscopic features in a workspace 40. The microscope 24 may be mounted on a support arm 30. The support arm 30 may be part of a robot-assisted positioning apparatus such that the microscope field of view 25 of the microscope 24 may be variously re-positioned. The system 10 includes at least one camera 80 coupled to the support arm 30 with the at least one camera 80 in signal communication 22 with a controller 20. In some examples, the system 10 includes a bracket coupling each of the at least one camera 80 with the support arm 30. In some example as shown, there are two cameras 80 in the system 10, in which the cameras are positioned above and spaced apart from the workspace 40.

Each of the at least one camera 80 is selected to have a camera field of view 85 that is at least larger than the microscope field of view 25 of the microscope 24. Preferably, each camera 80 has a horizontal camera field of view of about 56 degrees and a vertical camera field of view of about 46 degrees. Preferably, at any one time in use, a pair of the cameras 80 are provided to capture respective camera fields of view 85, in which the camera fields of view 85 overlap with one another. Preferably, the cameras 80 are respectively positioned and/or oriented such that the combined camera field of the view 85 overlaps or coincides with the microscope field of view 25. The camera 80 is preferably oriented to include at least a part of a workspace 40 that is simultaneously in the camera field of view 85 of the camera 80 and the microscope field of view 25. As used herein, the term “workspace” refers to a volumetric or three-dimensional space within which the user performs high-precision manipulation at a microscopic level, e.g., a three-dimensional space in which a position of a micromanipulator or a tool holder/tool is to be determined according to a method disclosed herein. For convenience and not to be limiting, the workspace 40 may be described in the form of an imaginary cuboid with linear dimensions in a range of about 40 centimeters (cm) by 40 cm.

Alternatively, in one example, the workspace 40 may be approximated by a space about 40 cm by 20 cm by 30 cm. In another example, the workspace 40 may be around 50 cm (width) by 30 cm (depth) by 20 cm (height). The combined camera field of view 85 of the pair of cameras 80 may be on the scale of tens of centimeters. This is a relatively large workspace compared to conventional vision systems designed for surgical assistance.

The system 10 is operable with as few as one camera 80. Preferably, the system includes two cameras 80 or three cameras 80. FIG. 1B schematically illustrates a non-limiting example in which three cameras 80 are mounted so that they are distributed about an objective lens 26 of the microscope 24 (the mountings are not shown to avoid obfuscation). For ease of reference, the objective lens 26 of the microscope 24 may serve to define a reference plane 27. It can be appreciated that the reference plane 27 need not always be parallel to the ground. The reference plane 27 may alternatively be defined as being normal to an optical axis 28 extending through a geometric center of the objective lens 26. In this embodiment, in operation, each of the cameras 80 are positioned above the reference plane 27. Each of the at least one camera 80 is positioned at a positive vertical displacement (V) relative to the reference plane 27. In other words, each one of the at least one camera 80 is preferably positioned further away from the workspace 40 than the objective lens 26 of the microscope 24.

Examples of the camera 80 include but are not limited to various optical sensors, such as motion capture cameras (MoCap), CMOS (complementary metal oxide semiconductor) cameras, CCD (charge-coupled device) cameras, TOF (time-of-flight) cameras, other position sensing detectors, or any combination thereof, etc. Preferably, each of the at least one camera 80 is a motion capture optical camera having an accuracy in a range of at least 400 microns, or preferably, an accuracy in a range of at least 300 microns. These are cameras for use in capturing relatively large motion, e.g., motion of the limbs of a walking subject. The one or more cameras 80 may be selected such that the workspace 40 is a three-dimensional space that is at least one order of magnitude smaller than a camera field of view 85 of the at least one camera 80. The camera field of view (or the combined camera field of view) 85 is configured to at least overlap the microscope field of view 25 of a microscope 24 for the same workspace 40. The camera 80 selected should have a sampling frequency/operating frequency that is substantially higher than the typical frequency of hand tremors. In some examples, the camera 80 has a sampling frequency of more than 180 Hertz (Hz). Preferably, in operation, each of the at least one camera 80 is configured to have a frequency of motion capture that is greater than 200 Hz or greater than about 200 Hz (e.g., at least about 200 frames per second).

The system 10 is particularly useful in various applications involving microscopic level precision work in the workspace 40, examples of which include but are not limited to microsurgical procedures such as neurosurgeries, optics manufacturing and testing, microchip manufacturing and testing, microbiological applications, etc. Physiological tremor of a hand may be defined as movement that is involuntary, approximately rhythmic, and roughly sinusoidal, with a peak-to-peak error that can exceed 29 microns and a frequency in a range of about 8 Hz to about 12 Hz. As an example, hand tremor may be about 50 microns or micrometers (μm) in any direction.

The conventional manipulator with tremor-counteracting means is bulky from the need to provide motors and actuators for counteracting hand tremors that may occur in any direction. The exemplary conventional manipulator 90 schematically shown in profile in FIG. 1 illustrates how it can block the user (e.g., the surgeon) from viewing the relatively small workspace. In particular, it is critical to maintain a clear line of sight between the microscope 24 and the working end of whatever manipulator the surgeon is using.

Proposed herein is a handheld instrument that addresses issues associated with conventional manipulators. To aid understanding, the following will describe one embodiment of the handheld instrument in the form of a micromanipulator 100 with interchangeable tools for surgical operations, but it will be understood that various embodiments of the handheld instrument may be provided to compensate or mitigate hand tremors in other applications. A wide variety of tools may be provided, including tools with operable or moving parts. Advantageously, as will be understood from the following description, the micromanipulator of the present disclosure has a more streamline and ergonomic profile such that various tools can be easily and releasably attached to a tool holder of the micromanipulator, and such that the attached tool can be operated by the same hand holding the micromanipulator, e.g., via a lever-like mechanism that is both connected to the tool tip and accessible by a thumb or finger of the hand holding the micromanipulator.

In some embodiments, the micromanipulator 100 may be provided with at least one marker 110 disposed or coupled on an exterior of the micromanipulator 100. Preferably, a plurality of the markers 110 are distributed along an exterior of micromanipulator 100. In some embodiments, the at least one marker 110 may include a three-dimensional (3D) marker, in other words, a marker having 3D features. As an example, the 3D marker may include visually distinctive features in each of three orthogonal dimensions such that information from each dimension may be distinct from other dimensions. In other embodiments, the at least one marker 110 may include two or more two-dimensional markers (i.e., markers having 2D features). As an example, the 2D marker may include visually distinctive features in two dimensions such that information from each dimension may be distinct from the other dimension. In other embodiments, the at least one marker 110 may include three or more one-dimensional markers (i.e., 1D markers). In some embodiments, the 1D markers may be active markers configured to illuminate one light emitting diode (LED) at a time very quickly or multiple LEDs for positional identification, akin to celestial navigation. Rather than reflecting light as in the case of passive markers, active markers are powered to controllably emit light. In embodiments employing LED-based markers 110, the LEDs and the cameras 80 may be frequency matched so that ambient noise and other lights in the operating theatre may be rejected as noise to the present system 10.

In use, a plurality of the markers 110 are distributed in different places on the exterior of the micromanipulator 100 such that the user may be free to use a variety of hand poses to hold and manipulate the micromanipulator 100, without having to worry about the visibility of any marker. It has been experimentally verified with a preferred embodiment that, with a plurality of 2D markers 110 distributed along the length of the micromanipulator 100, at least one of the plurality of markers 110 can be visible to the cameras 80 when the surgeon handles the micromanipulator 100 in the usual manner comfortable and familiar to the surgeon. The controller 20 is configured to determine a position and an orientation of the micromanipulator 100 from the images captured by one or all of the cameras 80. That is, the position and the orientation of the working end (tool tip) of the micromanipulator 100 relative to the workspace 40 can be tracked in real time. Prototypes have demonstrated a positional accuracy of about 10 microns.

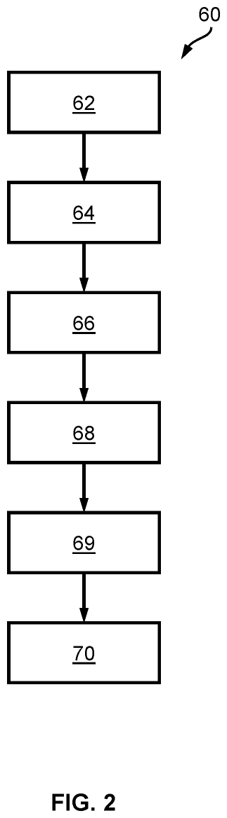

Referring to FIG. 2, in one embodiment, a method 60 of tremor mitigation includes identifying the markers 110 on the micromanipulator 100 from images captured by motion capture cameras 80 (62). Preferably, the method 60 includes operating each of the at least one camera 80 to capture at least about 200 frames per second. Preferably, each of the at least one camera 80 is selected from motion capture cameras with an accuracy of up to about 400 microns accuracy for capturing motion of the at least one markers 110 in an application where the accuracy required of the motion to be captured is about 10 microns. Preferably, the method 60 includes selecting one or more motion capture cameras with an accuracy of at least about 300 microns, for capturing motion that requires an accuracy of at least about 10 microns. Conventional thinking would not expect a camera with an accuracy in the range of 300 to 400 microns to be sufficiently sensitive to capture the tiny tremors in the range of 10 microns. Surprising, using a motion capture camera traditionally used for measuring relatively large movements (such as the swinging limbs of a walking or running subject) advantageously provide a relatively large coverage of the workspace 40 and, in conjunction with the markers 110, enable the tiny tremors to be determined with sufficient accuracy. The method 60 includes computing the positions of the markers 110 in at least three-dimensional coordinates (64). The three-dimensional coordinates of the markers 110 may be converted to tool tip coordinates (66). The conversion may include a relatively straightforward mathematical computation based on the specific geometry of the micromanipulator 100 and the interchangeable tool tip. The controller 20 may be configured to compute to compensate for hand tremor in an 8 Hz to 12 Hz frequency range (68). For example, if a 10 Hz tremor-induced motion is detected in one direction, the controller 20 may instruct the micromanipulator 100 to move the tool tip in an opposite direction by a distance of similar magnitude. The controller 20 may be configured to communicate with an actuator in the micromanipulator 100 to counteract hand tremor movements (70). For the sake of brevity, in the present disclosure, references to moving the tool tip may be understood as moving the tool holder relative to the micromanipulator body, the tool being coupled to and carried by the tool holder so as to present the tool tip 400 beyond the micromanipulator 100.

From the images captured by the cameras 80, the controller 20 is configured to detect motion of the micromanipulator 100, and in particular, to detect tiny movements associated with a frequency in a range typical from about 8Hz to about 12 Hz, inclusive of both 8 Hz and 12 Hz. A tremor-mitigating or compensating displacement (distance and direction) is calculated or otherwise determined by the controller 20 and communicated to an actuating mechanism in the micromanipulator 100. The compensating displacement refers to the displacement of a tool tip relative to the micromanipulator body (the micromanipulator body is handheld by the user when in use) which is intended to mitigate or compensate hand tremors of the user. That is, the controller 20 is configured to controllably actuate a tool holder of the micromanipulator 100 such that the positioning of the tool/tool tip (carried by the tool holder) has reduced variable deviations caused by hand tremors. The resulting effect is a steadier tool tip as well as more accurate positioning by the user, as tremor-related effects are mitigated in real time by the micromanipulator 100. All the while, the surgeon can use the micromanipulator 100 as though the micromanipulator 100 is a traditional surgical instrument, i.e., the surgeon does not need to modify surgical techniques solely for the sake of using the micromanipulator 100. Part of this advantage is rendered possible by the relatively slim and streamlined profile of the micromanipulator 100, such that the surgeon can essentially use familiar hand poses for holding and manipulating surgical instruments.

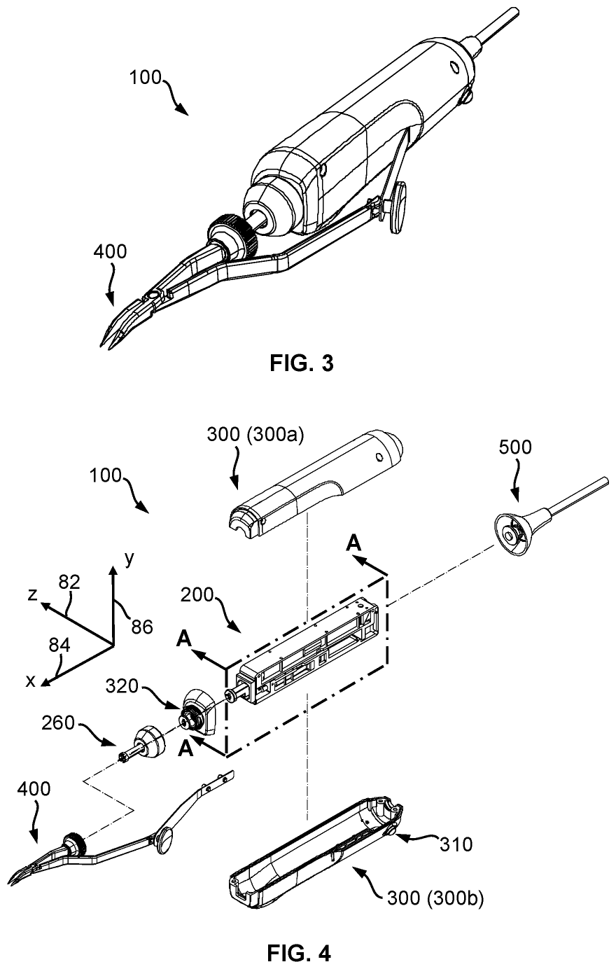

FIG. 3 is a perspective view illustrating an embodiment of the handheld instrument proposed herein, in an example of a micromanipulator 100 for surgical applications. The micromanipulator 100 is shown in an exploded view in FIG. 4. The micromanipulator 100 has high dexterity with no static singularity. The micromanipulator 100 includes a flexural frame 200 disposed in a housing 300. The micromanipulator 100 includes a tool holder 260 to receive or to be releasably coupled with an interchangeable or replaceable tool tip 400. For surgical applications, the tool tip 400 may be any one of a needle holder, forceps, scissors, etc. The micromanipulator 100 includes an electrical connector 500 for signal communication 22 and/or power communication with the controller 20. In some embodiments, the housing 300 may further include one or more waterproof or resistant seals 320 to prevent any ingression of liquid into the housing 300. Optionally, the housing 300 may include two mutually attachable housing portions 300a/300b with an exterior shaped to be handholdable by a user. As illustrated in the example of FIG. 3, the housing 300 can be conformed to be ergonomic for a good grip and comfortable handling.

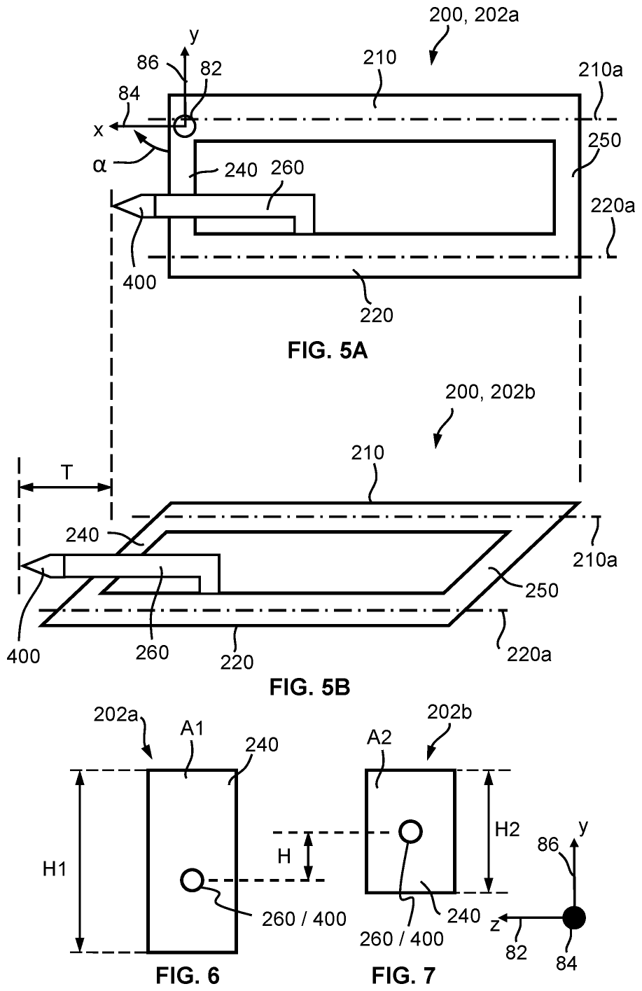

To aid understanding, FIGS. 5A and 5B illustrate a schematic diagram of the flexural frame 200. The flexural frame 200 includes a first frame portion 210, a second frame portion 220, a first connecting portion 240, and a second connecting portion 250 coupled in a quadrilateral configuration 202. The first connection portion 240 is pivotably coupled to the first frame portion 210 at one end and pivotably coupled to the second frame portion 220 at another end. The second connection portion 250 is pivotably coupled to the first frame portion 210 at one end and pivotably coupled to the second frame portion 220 at another end. The first frame portion 210 is pivotably coupled to the first connecting portion 240 at one end and pivotably coupled to the second connecting portion 250 at another end. The second frame portion 220 is pivotably coupled to the first connecting portion 240 at one end and pivotably coupled to the second connecting portion at another end. The first connecting portion 240 is disposed at a distal end of the micromanipulator 100 and the second connecting portion 250 is disposed nearer a proximal end of the micromanipulator 100. It may be appreciated that each of the respective coupling points or coupling surfaces between any pair of the first frame portion 210, the second frame portion 220, the first connecting portion 240, and the second connecting portion 250, may be about an arbitrary axis neighboring to or at a joint between the relevant portions of the flexural frame 200, and need not be at the respective extremities as illustrated. The flexural frame 200 may be formed in parts and connected together. Alternatively, and preferably, the flexural frame 200 may be formed as a unitary article. The actual dimensions of the flexural dimensions may be varied. Preferably, the flexural frame 200 has an elongate shape as illustrated.

For convenient reference, the first frame portion 210 may be described as being axially oriented or disposed in a first plane 210a. In a first frame state 202a (as shown in FIG. 5A), the second frame portion 220 may be disposed in a second plane 220a substantially parallel to and laterally spaced apart from the first frame portion 210. The first frame portion 210 is fixedly coupled to the housing 300. Responsive to the first connecting portion 240 being pushed in an axial direction 84 relative to the first frame portion 210, the second frame portion 220 moves such that the flexural frame 200 changes from a first frame state 202a (as shown in FIG. 5A) to a second frame state 202b (FIG. 5B). The second frame portion 220 is constrained by the first connecting portion 240 and the second connecting portion 250 to exhibit an angular displacement α, relative to a first axis 82, in addition to a translational displacement in the axial direction 84 relative to the first frame portion 210. The second frame portion 220 may be said to also be displaced along a third axis 86 (in a lateral direction orthogonal to both the first axis 82 and the axial direction 84) towards the first frame portion 210.

The tool holder 260 may be coupled to the second frame portion 220 so that a displacement of the second frame portion 220 relative to the first frame portion 210 results in a corresponding displacement of the tool holder 260 and of a tool tip 400 that is attached to the tool holder 260. That is, by bringing the flexural frame 200 from a first frame state 202a to a second frame state 202b, the tool holder 260 (and correspondingly the tool tip 400) may undergo a resultant displacement T in the axial direction 84 as well as a resulting displacement H along a lateral direction (along the third axis 86). It will be understood that, alternatively, the second connecting portion 250 may be pushed or biased to move in an opposing direction to the axial direction 84.

FIG. 6 schematically illustrates a view of the flexural frame 200 with a tool tip 400 attached thereto, in the first frame state 202a of FIG. 5A. FIG. 7 schematically illustrates a view of the flexural frame 200 with a tool tip 400 attached thereto, in the second frame state 202b of FIG. 5B. As illustrated, the “footprint” (as represented by an area A1 of the flexural frame in the first frame state 202a) can be reduced to a smaller footprint (as represented by an area A2) when the second frame portion 220 is actuated axially relative to the first frame portion 210. A smaller footprint means that the micromanipulator 100 will potentially present less blockage to a line of sight to the workspace 40. Advantageously, the physical dimensions of the housing 300 of the manipulator 100 may be determined by the physical dimensions of the quadrilateral frame 210 in the first frame state 202a (e.g., this may correspond to a non-operating state of the micromanipulator 100). In an example, the housing 300 may have a compact maximum diameter of around 24 mm and a weight of 62 grams, well within the acceptable range for handheld instruments.

Preferably, the flexural frame 200 forms a parallelogram such that, in the first frame state 202a, the first plane 210a is parallel to the second plane 220a. When the flexural frame 200 transitions from the first frame state 202a to the second frame state 202b, the first frame portion 210 and the second frame portion 220 remain substantially parallel throughout the transition. This means that the tool tip 400 essentially remains in the same orientation throughout this transition.

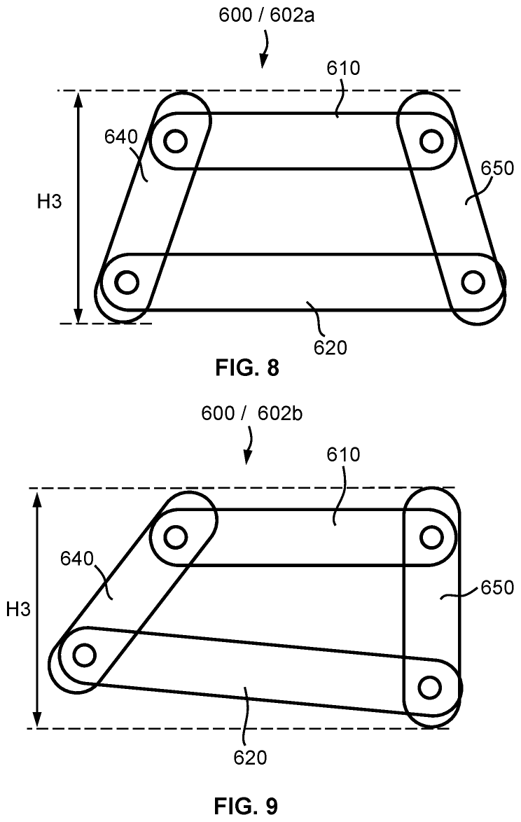

Another embodiment of the flexural frame is schematically illustrated in FIGS. 8 and 9. In this example, FIG. 8 illustrates a flexural frame 600 (an example of the flexural frame 200) in a first frame state 602a, and FIG. 9 illustrates the flexural frame 600 in a second frame state 602b. The flexural frame 600 includes a first frame portion 610; a second frame portion 620 spaced apart from the first frame portion 610; a first connecting portion 640, and a second connecting portion 650 spaced apart from the first connecting portion 640. The first connecting portion 640 is coupled to each of the first frame portion 610 and the second frame portion 620 via respective spaced apart pivotable couplings. Similarly, the second connecting portion 650 is coupled to each of the first frame portion 610 and the second frame portion 620 via respective spaced apart pivotable couplings. The pivotable couplings may be spring loaded such that the flexural frame 600 is biased to a non-operating frame state, for example, the first frame state 602a. In this example, the flexural frame 600 is not constrained to a parallelogram configuration. In addition to translation, the second frame portion 620 undergoes a rotational displacement relative to the first frame portion 610. That is, a translational displacement and a rotational displacement of an attached tool tip may be achieved. The tool holder may be connected to either the first connecting portion 640 or the second frame portion 620 such that by transitioning from the first frame state 602a to the second frame state 602b, the tool tip may undergo corresponding small displacements suitable for at least partially counteracting tremors. In this example, the largest footprint may correspond to a dimension H3 or a length of the second connecting portion 650.

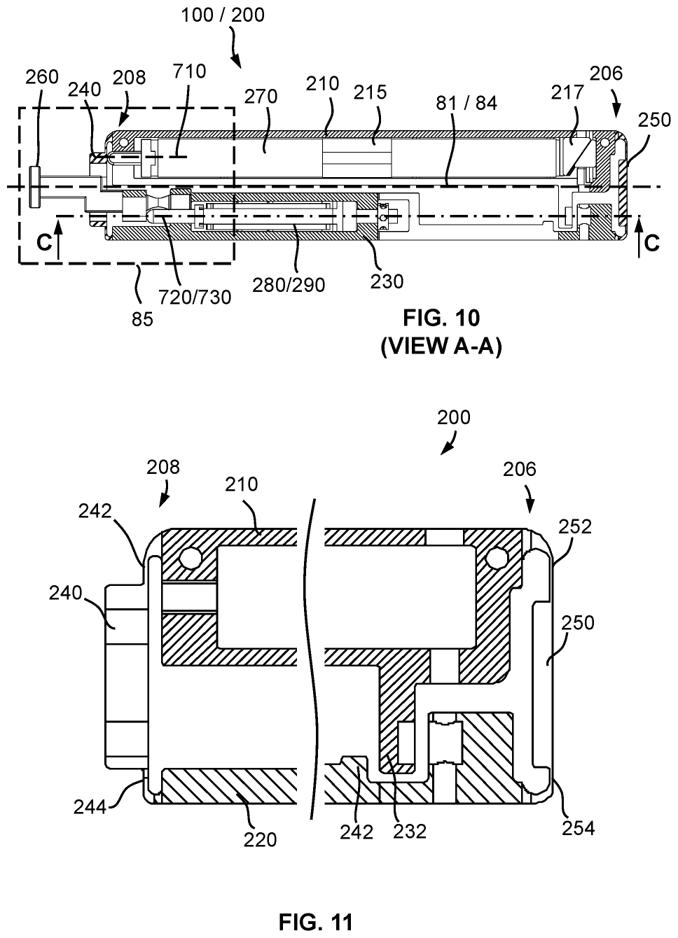

FIGS. 10 and 11 are partial cross-sectional view of a part of the micromanipulator 100 of FIG. 4, in which the micromanipulator includes an actuating mechanism based on the flexural frame 200 of FIG. 5A/Fig. 5B. The flexural frame 200 is coupled with a tool holder 260, which is in turn configured for attachment with a tool/tool tip 400. The first frame portion 210 may be described as elongated and generally defining an axial direction 84 extending from a first proximal end 206 to a first distal end 208. The first frame portion 210 may provide a first slot 215 in which a primary actuator 270 may be securely disposed. The primary actuator 270 is oriented axially with its actuating or working end near the first distal end 208 of the flexural frame 200. The first connecting portion 240 connects the first frame portion 210 and the second frame portion 220 at the distal end 208 of the flexural frame 200/first frame portion 210. The first connecting portion 240 couples the second frame portion 220 and the first frame portion 210 in respective resilient pivotable coupling. When the primary actuator 270 is in operation and actuates, the primary actuator 270 applies a linear pushing force on a part of the first connection portion 240, along the primary actuator axis 710 which may be substantially parallel with the axial direction 84. A first bias element 217 may be provided such that, when the primary actuator 270 is in a non-operating state, the flexural frame 200 is biased towards reverting back to the first frame state 202a. The second connecting portion 250 connects the first frame portion 210 and the second frame portion 220 at the proximal end 206 of the flexural frame 200/first frame portion 210. The second connecting portion 250 couples the second frame portion 220 and the first frame portion 210 in respective resilient pivotable coupling. The second frame portion 220 may be coupled to the first frame portion 210 only at the first connecting portion 240 and at the second connecting portion 250. The second frame portion 220 may be elongate and extends substantially in the axial direction 84 from the first proximal end 206 to the first distal end 208. The second frame portion 220 includes a support frame 230 in which a first secondary actuator 280 and a second secondary actuator 290 may be securely disposed. The support frame 230 may be carried by motion of the second frame portion 220 such that the support frame 230 is slidable along the axial direction relative to the first frame portion 210. The support frame 230 is disposed near the first distal end 208. The first secondary actuator 280 is operable to provide a linear pushing force on the tool holder 260 along the first secondary actuator axis 720. The second secondary actuator 290 is operable to provide a linear pushing force on the tool holder 260 along the second secondary actuator axis 730. In the first frame state 202a as shown in FIG. 10, the primary actuator axis 710 is parallel to the first secondary actuator axis 720 and the second secondary actuator axis 730. The primary actuator axis 710 and the pair of secondary actuator axes 720/730 are disposed on either side of a central axis 81 of the micromanipulator/flexural frame 100/200.

The first frame portion 210, the second frame portion 220, the first connecting portion 240, and a second connecting portion 250 together form the flexural frame 200. The flexural frame is resiliently biased to the first frame state 202a. In the first frame state 202a, the tool holder 260 is in a retracted position (e.g., as shown in FIG. 10, the retracted position of the tool holder 260 corresponds to the first frame state 202a of the flexural frame 200), relative to the first distal end 208. Actuation by any one or more of the actuators 270/280/290 displaces the tool holder 260 out of the retracted position. Owing to the resiliently biased configuration of the flexural frame 200, the tool holder 260 (and correspondingly the tool tip 400) will tend to return to the retracted position after it is displaced. The tool holder 260 (and the tool tip 400) may thus exhibit a vibration-like motion to mitigate hand tremors. Optionally but preferably, the flexural frame 200 is an integrally formed article or a unitary article, with flexural couplings 242/244/252/254 at the corners where the first frame portion 210 meets the first connection portion 240, the first connecting portion meets the second frame portion 220, the second frame portion 220 meets the second connecting portion 250, and the second connecting portion 250 meets the first frame portion, respectively. In some examples, the flexural frame 200 may be made laser cut from a single piece of lightweight metal, such as but not limited to aluminum. The flexural couplings 242/244/252/254 may each have a narrowed cross section, for example, a sheet-like structure, to form a resilient pivot between one of the frame portions 210/220 and a respective connecting portion 240/250, such that the frame portions 210/220 and the connecting portions 240/250 are resiliently displaceable and pivotable relative to one other.

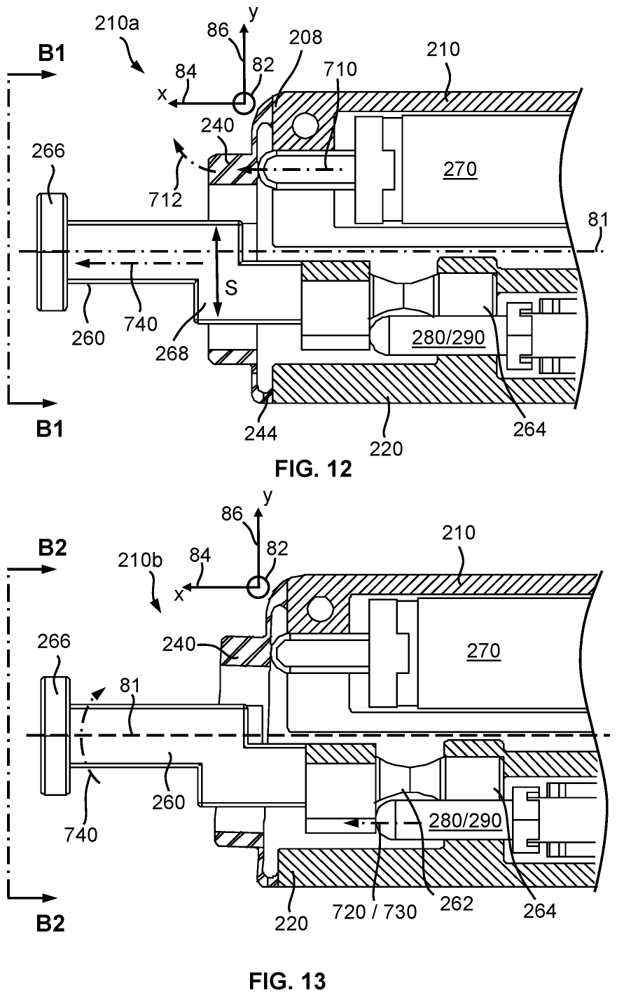

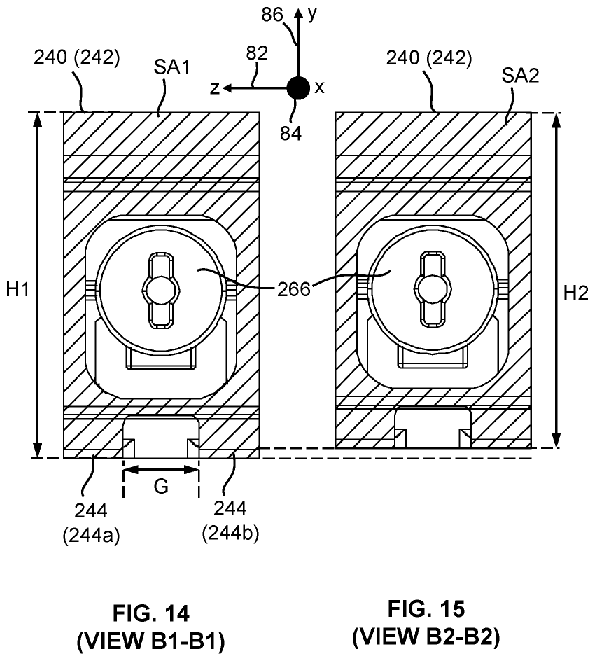

Reference will be made to FIGS. 12 to 19 which illustrate different views of the actuating mechanism in different states. FIGS. 12 and 14 illustrate the flexural frame 200 in a first frame state 202a, and FIGS. 13 and 15 illustrate the flexural frame 200 in the second frame state 202b. The primary actuator 270 (such as a linear displacement actuator) is coupled to the first frame portion 210. That is, the primary actuator 270 is coupled to the first frame portion 210 to provide a displacement relative to the first frame portion 210 or relative to the housing 300. The primary actuator 270 may be a piezoelectric linear actuator in abutment with the first connecting portion 240. The primary actuator 270 may be configured to bear on the first connecting portion 240 along a primary actuator axis 710.

The linear translation of the primary actuator 270 results in a moment pivoting the first connecting portion in an angular displacement 712 about a distal end 208 of the first frame portion 210. In the non-operating state (in this example, equivalent to the first frame state 202a), the primary actuator axis 710 is substantially parallel to the axial direction 84.

That is, when the primary actuator 270 pushes onto the first connecting portion 240 along the primary actuator axis 710, this causes the first connecting portion 240 to pivot relative to the first frame portion 210. Angular displacement 712 of the first connecting portion 240 translates into a translational displacement of the second frame portion 220 relative to the first frame portion 210, bringing the flexural frame 200 to a second frame state 202b as shown in FIG. 13. Angular displacement 712 of the first connecting portion 240 causes a displacement 740 to the second frame portion 220 and moving the tool holder 260 substantially in the axial direction.

Further, as shown in FIGS. 14 and 15, by transiting from the first frame state 202a to the second frame state 202b, the second frame portion 220 also moves parallel to a lateral axis 86 (y-axis) reducing a height of the quadrilateral frame 202 from height (H1) to height (H2). With the reduction in height of the quadrilateral frame 202, a footprint (SA1) of the flexural frame 200 can be reduced to a smaller footprint (SA2).

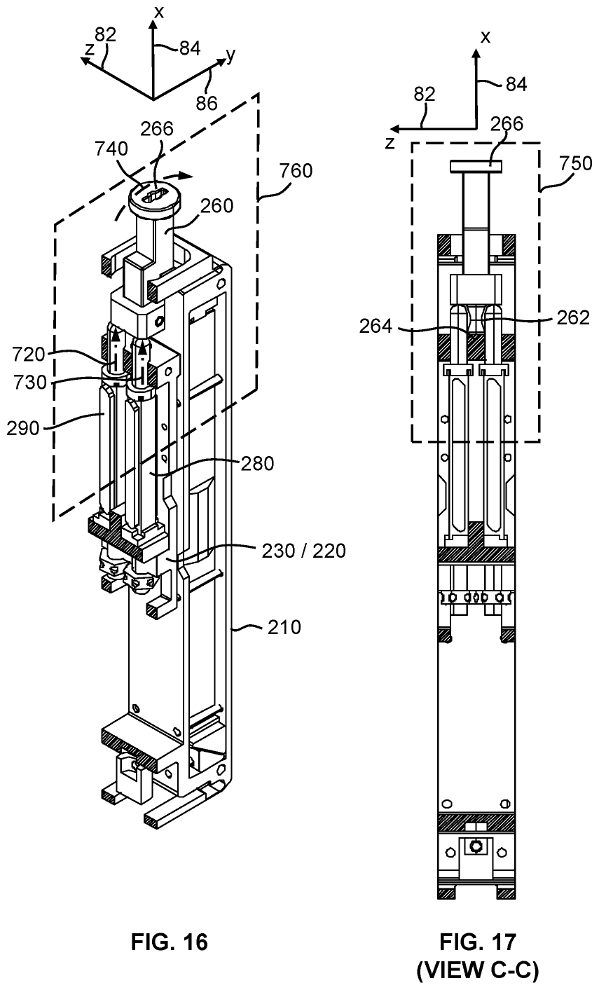

FIGS. 16 and 17 show part of the micromanipulator 100 with a part of the second frame portion 220 cut away to better to show the first secondary actuator 280 and the second secondary actuator 290. The plurality of secondary actuators 280/290 are preferably coupled fixedly to a support frame 230, in which the support frame 230 is part of the second frame portion 220. If the second frame portion 220 is displaced relative to the first frame portion 210, the second frame portion 220 carries the plurality of secondary actuators 280/290 along in the displacement of the second frame portion 220. Thus, the resultant displacement of the tool holder 260 (and the attached tool tip 400) would be a result of any actuation of either or both of the secondary actuators 780/790 and any actuation of the primary actuator 270.

Preferably, at least two secondary actuators 280/290 (such as two linear displacement actuators) are provided for each primary actuator 270 provided. The plurality of secondary actuators 280/290 coupled to the second frame portion 220 may be a pair of piezoelectric linear actuators, spaced apart from one another, and each in abutment with the tool holder 260. The secondary actuators 280/290 may each be configured to bear on the tool holder 260 along a respective secondary actuator axis 720/730 to pivot the tool holder 260. The secondary actuator axes 720/730 are parallel and non-coincident. In the non-operating state, each of the secondary actuator axes 720/730 may be substantially parallel to the axial direction 84. Preferably, each of the secondary actuator axes 720/730 is also parallel to the primary actuator axis 710. In an example where two secondary actuators 280/290 are provided, a first secondary actuator 280 and a second secondary actuator 290 are disposed in the flexural frame 200 such that the secondary actuators 280/290 are equidistant from the primary actuator 270. Preferably, the primary actuator 270 and each of the secondary actuators 280/290 are oriented in parallel, i.e., with respective actuating axes 710/720/730 in substantial alignment or parallel with one another.

In some embodiments, the tool holder 260 may be fixedly coupled to the second frame portion 220 via a connecting end 264 (FIG. 12). The tool holder 260 may include a neck 262 configured to allow bending of the tool holder 260 relative to the connecting end 264/the neck 262. With each of the secondary actuator 280/290 concurrently pushing onto the tool holder 260 along the respective secondary actuator axis 720/730 with equal displacement, the neck 262 pivots or bends in a first plane 760 (x-y plane), pivoting a tool interface 266 of the tool holder 260 relative to the second frame portion 220 and away from the central axis 81 (FIG. 10). By controllably actuating both of the secondary actuators 280/290 by an equal displacement, the tool/tool tip 400 can be provided with an angular displacement 740 in the first plane 760 about the connecting end 264/the neck 262.

Referring to FIGS. 17 to 19, if only one of the two secondary actuator 280/290 is actuated to push on the tool holder 260 in the axial direction 84, the neck 262 bends or pivots in an x-z plane 750 orthogonal to the x-y plane 760. Alternatively, the first secondary actuator 280 and the second secondary actuator 290 may be actuated with different displacements to pivot the tool interface 266 of the tool holder 260 in an opposing direction. Therefore, by controllably actuating one of the secondary actuators 280/290 or controllably actuating the secondary actuators 280/290 with different displacements, pivoting of the tool tip about the neck 262 (serving as a pivot) in the x-z plane 750 can be achieved. It may be appreciated that bending or pivoting at the neck 262 is amplified to result in a larger angular displacement 770 at the tool interface 266, this due to a moment arm (L) between the neck 262 and the tool interface 266.

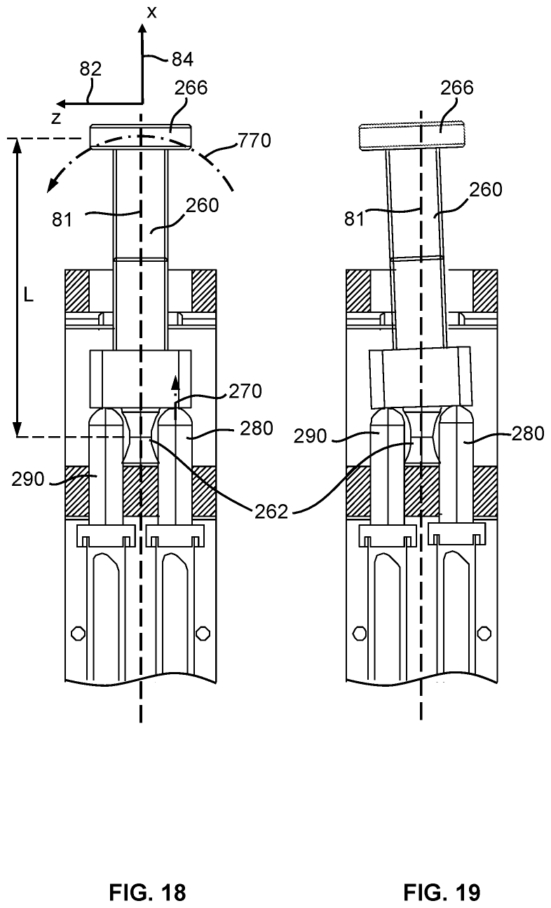

In some embodiments, the tool holder 260 is biased to a neutral position (e.g., the first frame state 202a) in which the tool interface 266 is substantially aligned with the central axis 81 as illustrated in FIG. 18. Upon actuation, tool interface 266 may assume an actuated position in which the tool holder 260 forms an oblique angle with the central axis 81 as illustrated in FIG. 19.

The controller 20 may be configured to be in signal communication 22 with each of the primary actuator 270, the first secondary actuator 280, and the second secondary actuator 290. The primary actuator 270 and each of the secondary actuators 280/290 are configured to actuate independently of one another, under the control of the controller 20. The tool tip 400 can be provided with three degrees of freedom relative to the micromanipulator housing 300. A position of the tool tip 400 may be controlled or maintained. As an example, the micromanipulator 100 may have an actuation stroke of as much as 300 μm.

Various modifications may be made to examples described above without going beyond the present claimed subject matter. For example, referring again to FIG. 11, in some embodiments, a first projection 232 may be provided on the first frame portion 210, and a second projection 242 provided on the second frame portion 240. The first projection 232 and the second projection 242 act as displacement limiters, such that when the first projection 232 abuts the second projection 242, the second frame portion 220 is prevented from further displacement in the axial direction 84 (relative to the first frame portion 210). In some embodiments, as shown in FIG. 12, the tool holder 260 may be formed with a step 268 to form an offset(S) relative to the second frame portion 220 such that (in a non-operating state) the tool/tool tip may be held nearer the central axis 81 of the micromanipulator 100. In some embodiments, as shown in the side view of FIG. 14, a gap (G) may be provided in the flexural coupling 244 to form flexure legs 244a/244b such that a stiffness/elasticity of the flexural coupling may be varied without changing the materials or the overall dimensions of the flexural frame 200.

All examples described herein, whether of apparatus, methods, materials, or products, are presented for the purpose of illustration and to aid understanding and are not intended to be limiting or exhaustive. Various changes and modifications may be made by one of ordinary skill in the art without departing from the scope of the invention as claimed.

1. A micromanipulator, comprising:
a first frame portion, the first frame portion defining an axial direction extending from a first proximal end to a first distal end;
a second frame portion;
a tool holder, the tool holder being coupled to the second frame portion;
a first connecting portion, the first connecting portion coupling the second frame portion and the first frame portion at the first distal end in resilient pivotable coupling; and
a second connecting portion, the second connecting portion coupling the second frame portion and the first frame portion at the first proximal end in resilient pivotable coupling, wherein the first frame portion, the second frame portion, the first connecting portion, and a second connecting portion form a flexural frame, the flexural frame being resiliently biased to a first frame state in which the tool holder is in a retracted position.

2. The micromanipulator according to claim 1, further comprising a primary actuator coupled to the first frame portion, the primary actuator being configured to provide a push force in the axial direction on the first connecting portion to angularly displace the first connecting portion about a first axis relative to the first frame portion, the first axis being orthogonal to the axial direction.

3. The micromanipulator according to claim 2, wherein a displacement of the second frame portion responsive to an actuation of the primary actuator results in a reduced footprint of the flexural frame.

4. The micromanipulator according to claim 2 or claim 3, further comprising:
a first secondary actuator coupled to the second frame portion, the first secondary actuator being a linear actuator having a first secondary actuator axis; and
a second secondary actuator coupled to the second frame portion, the second secondary actuator being a linear actuator having a second secondary actuator axis, wherein the primary actuator is a linear actuator having a primary actuator axis, and wherein the primary actuator axis, the first secondary actuator axis, and the second secondary axis are non-coincident with one another.

5. The micromanipulator according to claim 4, wherein the primary actuator axis, the first secondary actuator axis, and the second secondary axis are parallel to the axial direction at least when the flexural frame is in the first frame state.

6. The micromanipulator according to claim 4, wherein each of the first secondary actuator and the second secondary actuator is configured to provide a push force on the tool holder.

7. The micromanipulator according to claim 4, wherein the tool holder is angularly displaced relative to the second frame portion in response to actuation of at least one of the first secondary actuator and the second secondary actuator.

8. The micromanipulator according to claim 4, further comprising a controller, the controller being configured to be in signal communication with the primary actuator, the first secondary actuator, and the second secondary actuator, wherein the controller is configured to controllably actuate each of the primary actuator, the first secondary actuator, and the second secondary actuator.

9. The micromanipulator according to claim 1, wherein responsive to the first connecting portion pivoting relative to the first frame portion about a first axis, the second frame portion is displaced in the axial direction, wherein the first axis is orthogonal to the axial direction.

10. The micromanipulator according to claim 1, wherein responsive to an angular displacement of the first connecting portion relative to the first frame portion about the first axis, the second frame portion is displaced along a lateral direction towards the first frame portion, wherein the lateral direction is orthogonal to both the first axis and the axial direction.

11. The micromanipulator according to claim 1, wherein the flexural frame is integrally formed.

12. The micromanipulator according to claim 1, further comprising a housing, the housing being coupled to the first frame portion, wherein the micromanipulator is a handheld instrument.

13. The micromanipulator according to claim 12, further comprising at least one marker coupled to an exterior of the housing.

14. The micromanipulator according to claim 12, further comprising a plurality of markers coupled to an exterior of the housing, wherein each of the at least three markers are configured to emit light.

15. A system comprising:
a tool;
a micromanipulator, the micromanipulator including:
a first frame portion, the first frame portion defining an axial direction extending from a first proximal end to a first distal end;
a second frame portion;
a tool holder, the tool holder being coupled to the second frame portion, the tool being releasably attached to the tool holder;
a first connecting portion, the first connecting portion coupling the second frame portion and the first frame portion at the first distal end in resilient pivotable coupling;
a second connecting portion, the second connecting portion coupling the second frame portion and the first frame portion at the first proximal end in resilient pivotable coupling, wherein the first frame portion, the second frame portion, the first connecting portion, and a second connecting portion form a flexural frame, the flexural frame being resiliently biased to a first frame state in which the tool holder is in a retracted position relative to the first distal end;
a housing coupled to the first frame portion; and
at least one marker disposed on the housing; and
a controller, the controller being in signal communication with the micromanipulator, the controller being configured to determine a tremor-mitigating displacement for the tool based on images captured of the at least one marker.

16. The system of claim 15, wherein the micromanipulator further comprises:
a primary actuator coupled to the first frame portion, the primary actuator being configured to provide a push force in the axial direction on the first connecting portion to angularly displace the first connecting portion about a first axis relative to the first frame portion, the first axis being orthogonal to the axial direction; and
two secondary actuators coupled to the second frame portion, each of the two secondary actuators being a linear actuator with a respective secondary actuator axis, wherein the primary actuator axis and the respective second actuator axis are non-coincident with one another,
the controller being configured to control the primary actuator and the two secondary actuators to provide a tremor-mitigating displacement of the tool, and
a camera configured to capture the images, wherein the camera is a motion capture camera, and
wherein the at least one marker is a light emitting diode, and wherein the camera and the at least one marker are configured to match in frequency.

17. (canceled)

18. (canceled)

19. The system according to claim 15, comprising two cameras having a combined camera field of view, wherein the two cameras are mounted to a support arm above a workspace, and support arm being coupled with a microscope having a microscope field of view, and wherein the two cameras are oriented such that the combined camera field of view at least overlaps the microscope field of view.

20. A system comprising:
a tool;
a micromanipulator, the micromanipulator including:
a tool holder, the tool being releasably attached to the tool holder;
a housing; and
at least one marker disposed on the housing; and
at least one camera, the at least one camera being configured to capture images of the at least one marker; and
a controller, the controller being configured to be in signal communication with the micromanipulator, the controller being configured to determine a tremor-mitigating displacement for the tool based on the images of the at least one marker, wherein each one of the at least one camera is a motion capture optical camera having an operational accuracy of at least 400 microns, and wherein the tremor-mitigating displacement is determined to an accuracy of 10 microns and
wherein the controller is configured to determine a position of the tool holder in a workspace, the workspace being a three-dimensional space that is at least one order of magnitude smaller than a camera field of view of the at least one camera.

21. (canceled)

22. The system according to claim 20, the system further comprising a microscope having a microscope field of view, wherein the at least one camera having a combined camera field of view configured to at least overlap the microscope field of view, wherein each one of the at least one camera is positioned further away from the workspace than an objective lens of the microscope.

23. The system according to claim 20, wherein the at least one marker is a light emitting diode, and wherein the at least one camera and the at least one marker are configured to match in frequency.

24. (canceled)
